(19) United States
(12) Patent Application Publication
(10) Pub. No.: US 2009/0149838 A1
(43) Pub. Date: Jun. 11, 2009

(54) Subcutaneous Implant System

(76) Inventor: David C. Cassada, Knoxville, TN (US)

Correspondence Address: LUEDEKA, NEELY & GRAHAM, P.C., P O BOX 1871, KNOXVILLE, TN, 37901, US

(21) Appl. No.: 11/953,368

(22) Filed: Dec. 10, 2007

(51) Int. Cl. A61M 31/00

(52) U.S. Cl. 604/890.1

(57) ABSTRACT

Implantable subcutaneous drug delivery devices for treating chronic nerve pain, and methods of use thereof, are disclosed. The device includes a reservoir having a septum, and a flexible tube in fluid flow communication with the reservoir, with at least a portion of the tube having a plurality of sidewall apertures. The device is implanted such that the apertures are disposed proximal to the desired tissue for contacting the desired tissue with medicine, and the reservoir is disposed in an unobtrusive location distal from the desired tissue. Medicine is periodically and repeatedly percutaneously charged to the device via a cannula introduced from outside the body.

### TECHNICAL FIELD

The present disclosure is related to the field of wholly subcutaneous implantable drug delivery devices for the delivery of a drug directly to a desired tissue site, and methods thereof.

### BACKGROUND

Many aspects of human daily function depend upon use of the hands and arms to manipulate the immediate environment. Although it can be said for peripheral nerves at all locations, injury to the major nerve trunks of the brachial plexus (arms) may result not only in loss of critical human function, but also severe, relentless pain that is generally refractory to systemic or even parenteral drugs, including the strongest narcotics. Chronic nerve injury with and without and healing, may result in a constant and inexpressibly painful set of impulses experienced by the patient.

Surgery may be directed at sites of nerve injury from compression or traction, or any manor of nerve injury short of a complete transection, and there are a myriad of operations designed to decompress both peripheral nerves and the spinal cord. However, even after such surgery, pain at the nerve site may persist for an extended period of time.

It is also generally accepted that high dose narcotics can reduce nerve pain, however the systemic effects of such drugs may have long term disadvantages.

Newer medications directed at nerve pain alone demonstrate inconsistent effectiveness, and also may have systemic effects not tolerated by all patients. Accordingly, improvement is desired in the treatment of chronic nerve pain.

The disclosure relates to improvements in delivery of medicine via percutaneously accessible implants suitable for long-term use.

### SUMMARY

In one embodiment, the disclosure relates to an in-situ nerve drug delivery system located entirely within the body of a user for repeatedly and periodically delivering medicine to a desired nerve site. The system includes a delivery conduit located within the body of the user and below a skin portion of the body of the user, and configured for delivering medicine to the desired nerve site. A medicine introduction site is located within the body of the user and below a skin portion of the body of the user. The introduction site has a first portion in flow communication with the delivery conduit and a second portion in flow communication with the first portion. The second portion includes an interface for fluidly isolating the introduction site from the body of the user. The interface may be periodically and repeatedly percutaneously accessible via a cannula introduced from outside the body for periodically and repeatedly introducing the medicine to the introduction site for travel from the second portion to the first portion and the delivery conduit for delivery to the desired nerve site.

In another embodiment the disclosure provides a nerve drug delivery device. The device includes a reservoir having a volume and an elastomeric septum. The reservoir is in fluid flow communication with a tapered catheter tube having a reservoir end and a distal end. The distal end of the tube has a diameter that is less than the diameter of the reservoir end of the tube and includes a plurality of sidewall apertures. The device is subcutaneously disposed in a patient, and the sidewall apertures of the distal end are disposed proximal to a desired nerve bundle, while the reservoir is disposed at a subcutaneous access site for facilitating access to the septum with an externally introduced cannula in fluid flow communication with a source of medicine. The device may be charged with the medicine via the cannula, and the subcutaneous access site may be distal from the desired nerve bundle.

Other aspects of the disclosure relate to methods for treating chronic pain. For example, in one embodiment, the disclosure relates to a method for treating chronic nerve pain in a mammal. In accordance with the method, nerve tissue within the mammal that is associated with the chronic pain is determined. A delivery device is provided for introducing medicine to the nerve tissue. The device includes a reservoir and a septum in one-way flow communication with a first portion of the reservoir. The septum is configured for introducing medicine into the reservoir. A flexible elongate conduit having a proximal portion in one-way flow communication with a second portion of the reservoir is provided for evacuating the introduced medicine from the reservoir. A distal portion of the conduit is configured for releasing medicine evacuated from the reservoir. The delivery device is implanted within a patient so that the septum is disposed beneath and closely adjacent a region of skin of a patient to provide a subcutaneous access site. The distal end of the conduit is located proximate the determined nerve tissue for releasing medicine to the nerve tissue. The method may also include the step of providing an external cannula that is in flow communication with a source of medicine, and extending a distal end of the cannula through the skin of the patient and the elastomeric septum. Medicine may be released from the source of medicine through the cannula into the reservoir and sufficient flow pressure may be applied so that medicine in an amount effective to provide pain relief may flow from the reservoir to the distal end of the conduit and may release proximate the determined nerve tissue. The method may further include monitoring pain experienced by the mammal and determining when the pain may be sufficient to indicate the need for additional medicine to be delivered to the determined nerve tissue. Medicine may be repeatedly introduced into the reservoir in response to monitored pain experienced by the mammal as desired.

Accordingly, the present disclosure advantageously provides implantable drug delivery systems that may be periodically and repeatedly charged with medicine for treating chronic nerve pain over an extended period of time, as well as methods suitable for the treatment of chronic nerve pain. It has been observed that the systems and methods disclosed herein advantageously enable treatment of chronic pain and overcome disadvantages with prior treatment devices and methods. For example, one advantage relates to the provision of systems that are wholly subcutaneous, and are substantially shielded from visual observation by a casual observer. Wholly subcutaneous systems, such as disclosed herein, may also be less prone to infection.

It has been further observed that the system may remain functional in the body of a patient for extended periods of time, such as for at least a year, without removal of the system from the patient.

The simple construction and operation also advantageously avoids the need for any electrical power or moving parts that might malfunction.

Additional objects and advantages of the disclosure will be set forth in part in the description which follows, and/or can be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1 is an illustrative diagram of one embodiment of a drug delivery implant according to the present disclosure.

FIG. 2 is an illustrative diagram showing an exploded view of an embodiment of a drug delivery implant.

FIG. 3 is an illustrative diagram showing a low-profile reservoir.

FIG. 4 is an illustrative diagram of an alternative embodiment of a drug delivery implant of the present disclosure.

FIG. 5 is an illustrative diagram showing steps in a method for using a drug delivery implant in a patient's body in accordance with the present disclosure.

FIG. 6 is a cross-sectional diagram of a patient's arm showing a desired location for a portion of the implant.

### DESCRIPTION OF THE EXEMPLARY EMBODIMENTS

The present disclosure will now be described in the more limited aspects of preferred embodiments thereof, including various examples and illustrations of the formulation and use of the present disclosure. It will be understood that these embodiments are presented solely for the purpose of illustrating the invention and shall not be considered as a limitation upon the scope thereof.

With reference now to FIG. 1, one embodiment of a percutaneously accessible drug delivery implant device 10 in accordance with the present disclosure is shown. The device 10 may have a medicine introduction site such as a medicine reservoir 12 disposed at one end of the device 10, and a delivery conduit configured for delivering medicine to a desired nerve, such as a flexible catheter tube 14, in fluid flow connection with the reservoir 12.

The tube 14 may have a reservoir end 16 that is proximal to the reservoir 12, and a distal end 18 that is distal from the reservoir 12. At least a portion of the distal end 18 of the tube 14 may have plurality of sidewall apertures 20 configured for releasing the medicine to a desired tissue. The apertures 20 may range in diameter from about 10 μm to about 0.1 mm.

In some embodiments of the present disclosure, the tube 14 may be a flexible elastomeric catheter tube, or any other type of flexible tubing suitable for implantation in a mammalian body as a delivery conduit for a fluidic medicine. A suitable tube 14 may be made from a silicone elastomer, such as SILASTIC available from Dow Corning Corporation, or a cross-linked polydimethylsiloxane polymer. In some embodiments, the tube 14 may be tapered from the reservoir end 16 to the distal end 18, such that the diameter of the tube 14 at the reservoir end 16 is greater than the diameter of the tube 14 at the distal end 18, with at least a portion of the distal end 18 of the tube 14 having a plurality of the sidewall apertures 20.

In an alternative embodiment, the tube 14 may have a constant diameter. In a further alternative embodiment, two tubes of different diameters may be conjoined in a fluidic connection. Suitable tubing diameters may range from about 0.10 mm to about 6.00 mm.

One example of a desirable flexible tube 14 may be a section of a silastic lumbar drain, trimmed to a suitable length. In an alternative embodiment, the tube 14 may incorporate a coaxial metal rib or filament (not shown) longitudinally disposed within a tube wall for preventing fracturing or breakage of the tube 14 during implantation, use, or removal of the device 10.

The reservoir 12 may be a titanium reservoir having a fixed volume. A suitable volume of the reservoir 12 may range from about 0.5 mL to about 25 mL. The reservoir 12 may be made from any suitable material, such as a polymeric material or a non-ferrous metal, but titanium may be especially preferred as it is believed that the titanium may aid in reducing infection. It may be especially desirable that no ferrous material be used in the reservoir, so that MRI imaging may continue to be used with the patient.

Another desirable characteristic of the reservoir 12 may be a low-profile. In this context, “low-profile” means that a height dimension of the reservoir 12 may be substantially less than a length and/or a width dimension of the reservoir 12. A suitable height dimension of the reservoir 12 may range from about 0.25 cm to about 3.00 cm, with about 1.00 cm being particularly suitable, while suitable length and/or width dimensions may range from about 0.50 cm to about 10.00 cm, with about 2.50 cm to about 6.50 cm being particularly suitable.

The reservoir 12 may further include a septum 22. The septum 22 may be an elastomeric interface for fluidly isolating the reservoir 12 from the rest of a patient's body. The septum 22 may be periodically and repeatedly percutaneously accessible via a cannula introduced from outside the patient's body for periodically and repeatedly introducing and refilling the reservoir 12 with a medicine. The septum 22 may act as a one-way fluid flow valve to prevent the medicine from leaking into body tissue adjacent the reservoir 12. A suitable elastomeric septum 22 may be made from SILASTIC, available from Dow Corning Corporation, or a cross-linked polydimethylsiloxane polymer.

In one embodiment, the reservoir 12 may be configured to have a first portion in fluid flow communication with the tube 12, and a second portion in flow communication with the first portion. The second portion may include an interface for fluidly isolating the introduction site from the body of the user. The interface may be the septum 22.

Referring now to FIG. 2, an exploded view of an alternate embodiment of a drug delivery implant 10 shows the reservoir 12 having the septum 22, and a nipple 24 for the attachment of a first flexible tube 26 having a first diameter. The first diameter tube 26 may have a first end 28 and a second end 30. The first end 28 of the first diameter tube 26 may fit over the nipple 24, and a clamp 32 may secure the first end 28 of the first diameter tube 26 to the nipple 24, providing a fluid flow connection between the reservoir 12 and the first diameter tube 26.

The second end 30 of the first diameter tube 26 may be fit over a first end 36 of a coupling member 34. The coupling member 34 may be a relatively short hollow tube made of a sufficiently durable material, such as plastic or metal, for forming a secure end-to end fluid flow connection between two pieces of tubing. In one embodiment, the coupling member 34 may be a reducing coupling member, such that sections of tubing having two different diameters may be securely conjoined in a fluid flow connection.

A first end 42 of a second flexible tube 40 having a second diameter that is less than the first diameter of the first diameter tube 26 may be fit over a second end 38 of the coupling member 34, providing a secure fluidic connection between the first diameter tube 26 and the second diameter tube 40. In one embodiment, it may be desirable to externally secure the second end 30 of the first diameter tube 26 and the first end 42 of the second diameter tube 40 to the coupling member 34 with a clamp or other affixing means. The clamp may be titanium, or other suitable non-ferrous material.

The second diameter tube 40 may have a tip 44 that is distal from the first end of the second diameter tube 40. The tip 44 may be open-ended or sealed. At least a portion of the second diameter tube 40 proximal to the tip 44 may have a plurality of sidewall apertures 46, which may be substantially similar to the previously described apertures 20. The diameter of the apertures 46 may range from about 10 μm to about 0.1 mm. The plurality of apertures 46 may extend from the tip 44 toward the reservoir 12, and may be present on a suitable length of the tube 40 for treatment of a desired tissue, depending on the size of the area to be treated.

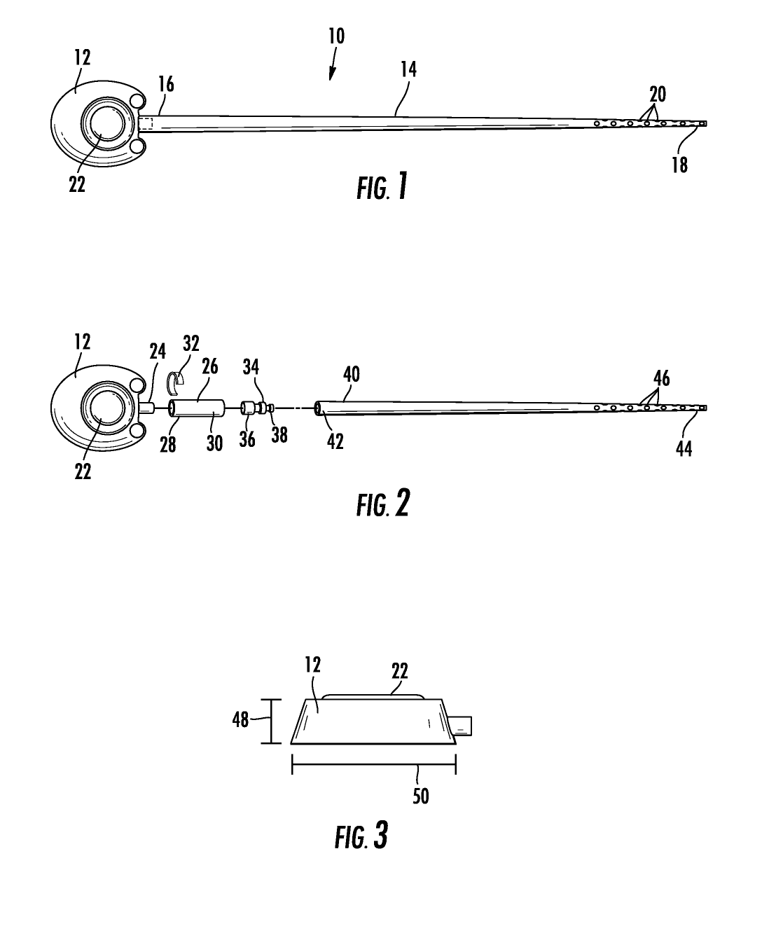

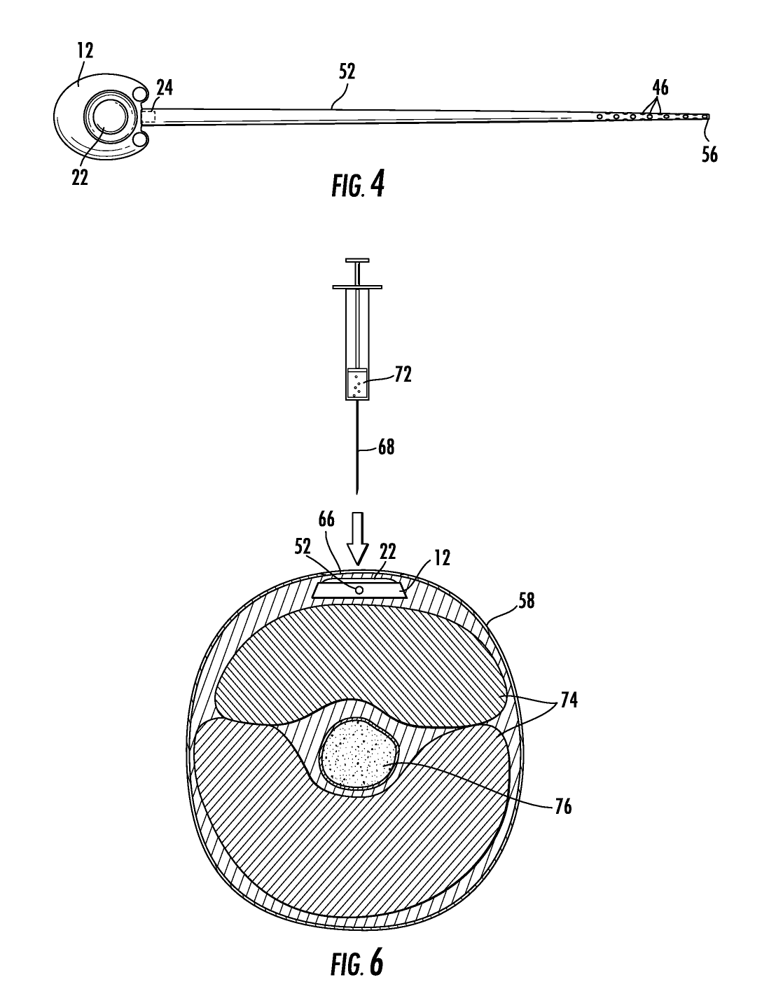

With reference now to FIG. 3, a low-profile reservoir 12 is illustrated. Such a reservoir may have a height dimension 48 that is substantially less than a width dimension 50. In one embodiment, the reservoir 12 may be substantially circular and the width dimension 50 may be a diameter of the reservoir 12.

FIG. 4 illustrates another embodiment of a drug delivery implant device 10 according to the present disclosure. The reservoir 12 may be fluidly connected, as described above, to a tapered flexible tube 52. The tube 52 may have a first diameter at a reservoir end 54 that is greater than a second diameter at a tip 56. At least a portion of the tip 56 end of the tube may have a plurality of sidewall apertures 46. This embodiment provides a simpler construction for the device 10, which may reduce both production cost and the risk of device 10 breakage or disconnection during use, implantation, or removal.

The foregoing embodiments have been shown configured from readily available components. However, in another preferred embodiment, the entire implant 10 is preferably formed to be of one-piece construction, such as of molded plastic or the like. Alternatively, the titanium reservoir may be coated with a polymeric or elastomeric coating, and the coated reservoir may be seamlessly affixed to the delivery conduit, providing an essentially one-piece construction.

In any of the forgoing embodiments, the device may have a coating for preventing or lessening infection or immune response in the adjacent tissue. Suitable coatings may include silver or silver-based coatings.

It may be especially desirable to discourage tissue ingrowth into the sidewall apertures, and therefore a suitable coating may be applied to at least a portion of the distal end of the tube for deterring tissue ingrowth. Alternatively, the material selected for the flexible tubing may have an inherent characteristic of deterring tissue ingrowth. Such materials or coatings may include coatings having hyaluronidase inhibitors, coatings having hyaluronidase enzymatic proteolytic chemistry, or coatings having a dilute papain enzymatic action.

The present disclosure also provides methods of using percutaneously accessible implant systems as described herein for providing long-term relief from chronic nerve pain in mammalian patients. The long-term pain relief may be provided by periodically and repeatedly refilling the implant with a suitable medicine. The implant may remain fully enclosed within a patient's body during the entire term of use, which may range from about 1 day to about the end of the patient's lifespan. A desirable term of use for the implant may range from about one week to about 50 years, with a further suitable example ranging from about 1 year to about 25 years.

The physician may locate the source of the pain before installing the implant in a patient. It is desirable that the source of pain be accurately located in order for the patient to receive the most pain-relief benefit from the implant. A patient experiencing chronic nerve pain may verbally identify the location of the pain to the physician. The physician may also utilize the patient's prior medical history, imaging diagnostic tests, such as MRI or CT scans, or any other suitable diagnostic tests, in order to ascertain the location of the nerve tissue causing the chronic pain. In some embodiments, the desired nerve tissue may be a peripheral nerve bundle including, but not limited to, the brachial plexus.

In alternate embodiments, the desired nerve tissue may be a nerve root, such as a lumbar nerve root. In such an embodiment, placement of the device may be achieved through exposure of the soft tissues adjacent the nerve root for disposing the distal end of the catheter tube. The catheter may then be tunneled to a non-obtrusive access site near the midsection, such as over a tendinous muscle or the iliac crest, below the beltline. The reservoir may be affixed to the reservoir end of the catheter and then implanted, septum side out, at the access site. The device may periodically and repeatedly be filled with medicine for alleviating the chronic nerve pain.

In further embodiments, the apparatus and methods disclosed herein may be routinely used for treatment of post-thoracotomy syndrome and non-entrapped dermatomal peripheral neuropathy, as well as any syndrome for chronic pain along the nerve roots at the axial skeleton, excluding intrathecal locations. Accordingly, the catheter tube of the present device may be suitable for disposition proximal to epidural locations, but may not be suitable for disposition within the dural sac.

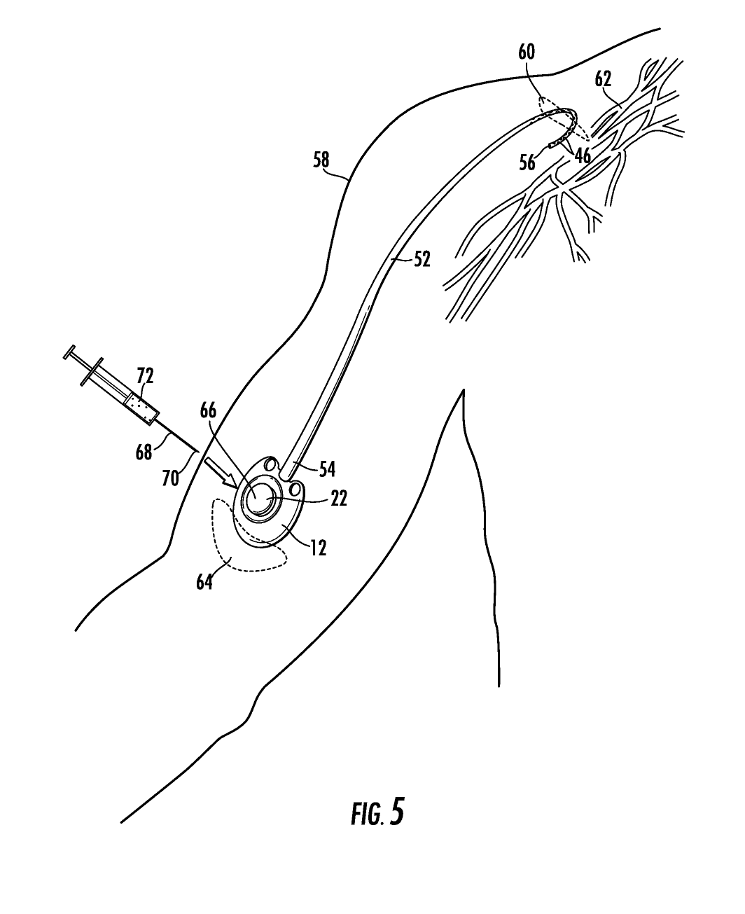

With reference now to FIG. 5, the device 10 may be implanted beneath a patient's skin 58. An incision 60 may be made near the identified source of nerve pain, which may be a peripheral nerve bundle 62. The tip 56 of the flexible tube 52, having the region of sidewall apertures 46, may be positioned adjacent to or on top of the desired nerve bundle 62, and may be shaped in such a fashion as to provide an effective amount of medicine from the apertures 46 to the nerve bundle 62. At least a portion of the tube 52 may optionally be sutured into position. The reservoir end 54 of the tube 52 may then be passed or threaded under the patient's skin 58 to a second incision site 64. The second incision site 64 may be on the patient's arm or another unobtrusive location, such that the reservoir 12 with the septum 22 facing outward may be disposed beneath and closely adjacent a region of skin of the patient to provide a subcutaneous access site 66. In one embodiment, the second incision site 64 and resultant access site 66 may be adjacent to the bicepital aponeurosis on the anterior, mid-upper arm.

The reservoir 12 may then be affixed to the reservoir end 54 of the tube 52, with the septum 22 facing out toward the surface of the skin 58. The reservoir 12, with the septum 22 properly positioned, having been affixed to the tube 52 may then be tucked under the patient's skin 58 at the second incision site 64. The first and the second incision sites 60 and 64 may then be sutured. The percutaneously accessible access site 66 may then be used for periodically and repeatedly refilling the reservoir 12 with medicine through an externally introduced cannula 68. The access site 66 may not be visible after healing of the incisions, and may only be detectable by touch, ultrasound, or other medical imaging technique.

In order to charge the implant with medicine, at least a portion of a distal (tip) end 70 of an external cannula 68 in fluid flow communication with a source of medicine 72 is passed through the patient's skin 58 and dermal tissue to penetrate the septum 22. Fluid flow pressure may be applied to the medicine 72, causing medicine 72 to flow through the cannula 68 into the reservoir 12, and from the reservoir 12 into the tube 52. The pressure may be sufficient to force an effective amount of the medicine 72 from the apertures 46 to contact the desired nerve tissue 62 and provide pain relief.

In one embodiment, the cannula 68 may be a syringe filled with medicine 72 and having a hypodermic needle. In another embodiment, the cannula 68 may be removably attached to a source of medicine 72.

After the device 10 has been charged with medicine 72, it may be desirable to flush the implant with a saline solution. Without removing the tip of the cannula from the reservoir, the source of medicine 72 may be removed from the cannula, and a source of saline (not shown) may be affixed. Fluid flow pressure may be applied to the saline to flush any remaining medicine 72 in the reservoir 12 and the tube 52 out the apertures 46 to contact the desired nerve tissue 62.

FIG. 6 shows a cross-sectional diagram of an implanted device showing an embodiment of the access site. The reservoir 12 is disposed beneath the skin 58, with the septum 22 facing the exterior of the patient's body. The tube 52 may be disposed beneath the skin 58 and above any muscle 74 or bone 76 tissue. The reservoir 12 may be charged with medicine by piercing the skin 58 and the septum 22 with a cannula 68 in fluid flow communication with a source of medicine 72, such that as fluid flow pressure is applied to the medicine 72, the medicine 72 passes through the cannula 68 into the reservoir 12. The septum 22 may prevent backflow of the medicine 72 or saline into the tissue proximal to the access site 66 after removal of the cannula 68.

The device 10 may periodically and repeatedly be charged with medicine in an amount effective to provide pain relief in the manner described above to treat chronic nerve pain as determined by the physician. The patient may be monitored by the physician to determine an effective amount of pain medicine. The amount of medicine charged to the device 10 may be increased or decreased depending on the amount of pain the patient may continue to experience, or the patient's tolerance to the medicine. The physician may determine when the amount of pain experienced by the patient may be sufficient to indicate the need for additional medicine to be delivered to the desired nerve tissue. Accordingly, it may be desirable to select a medicine that has a longer therapeutic effect to minimize the time period between implant charging.

Suitable medicines for treating chronic nerve pain via an implant as described herein may include steroids, such as dexamethasone, and/or local anesthetics such as bupivicaine, lidocaine, and the like. In some embodiments, doxepin or opiate-class drugs may also be used with the implants described herein.

Suitable amounts of medicine effective for treating chronic nerve pain may range from about 0.5 cc up to a continuous drip for an initial therapeutic treatment. The medicine may be provided in concentrations ranging from about 1 nanogram per cc to about 10 g per cc, where the concentration of medicine may depend on the potency of the specific medicine used and the severity of the pain experienced by the patient. The reservoir may be charged on a regularly scheduled basis, or it may be recharged as needed, as determined by the physician monitoring the patient's pain.

In the preceding examples, the device may be implanted using open or invasive surgical methods. In alternative embodiments, the device may also be implanted by non-invasive surgical methods. For example, the device may be implanted using fluoroscopic or CT guidance for placement. Using a commonly known breakaway sheath and wire system, also known as the Seldinger technique, a wire may be positioned at the desired nerve site, and then the wire may be exchanged for the catheter tube. In this manner placement of the implant may be achieved without making a large incision, but a relatively small puncture.

Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the invention being indicated by the following claims.

The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentee does not intend to dedicate any disclosed embodiments to the public, and to the extent any disclosed modifications or alterations may not literally fall within the scope of the claims, they are considered to be part hereof under the doctrine of equivalents.

What is claimed is:

1. An in-situ nerve drug delivery system for repeatedly and periodically delivering medicine to a desired nerve site within the body of a user, the system comprising:
a delivery conduit located entirely within the body of the user and below a skin portion of the body of the user, and configured for delivering medicine to the desired nerve site; and
a medicine introduction site located entirely within the body of the user and below a skin portion of the body of the user, the introduction site having a first portion in flow communication with the delivery conduit and a second portion in flow communication with the first portion, the second portion including an interface for fluidly isolating the introduction site from the body of the user and being periodically and repeatedly accessible via a cannula introduced from outside the body for periodically and repeatedly introducing the medicine to the introduction site for travel from the second portion to the first portion and the delivery conduit for delivery to the desired nerve site.

2. The system of claim 1, wherein the conduit comprises a silicone elastomer conduit.

3. The system of claim 2, wherein at least a portion of the conduit further comprises a metal rib longitudinally disposed within the conduit.

4. The system of claim 1, wherein the conduit comprises a proximal portion adjacent the first portion of the introduction site and a distal portion adjacent the desired nerve site, wherein the distal portion comprises a plurality of sidewall apertures.

5. The system of claim 1, wherein the conduit is tapered from the proximal portion to the distal portion.

6. The system of claim 1, wherein the introduction site comprises a fixed-volume reservoir having an interface comprising an elastomeric septum through which a portion of the cannula may pass for introducing the medicine via the cannula.

7. The system of claim 1, wherein the system may remain in the body of the user for an extended time period of more than a year.

8. A nerve drug delivery device, comprising a reservoir having a volume and an elastomeric septum, the reservoir being in fluid flow communication with a tapered catheter tube having a reservoir end and a distal end, wherein the distal end of the tube has a diameter less than the diameter of the reservoir end of the tube, and wherein the distal end has a plurality of sidewall apertures, the device being subcutaneously disposed in a patient, wherein the sidewall apertures of the distal end are disposed proximal to a desired nerve bundle, and the reservoir is disposed at a subcutaneous access site for facilitating access to the septum with an externally introduced cannula in fluid flow communication with a source of medicine, wherein the device is charged with the medicine via the cannula, and wherein the access site is distal from the desired nerve bundle.

9. The device of claim 8, wherein the reservoir comprises a low-profile reservoir.

10. The device of claim 8, wherein the catheter tube comprises a silicone elastomer catheter tube.

11. A method for treating chronic nerve pain in a mammal, the method comprising the steps of:
(a) determining nerve tissue within the mammal that is associated with the chronic pain;
(b) providing a delivery device including a fixed volume reservoir, a septum in one-way flow communication with a first portion of the reservoir and configured for introducing medicine into the reservoir, a flexible elongate conduit having a proximal portion in one-way flow communication with a second portion of the reservoir and configured for evacuating the introduced medicine from the reservoir, and a distal portion configured for releasing medicine evacuated from the reservoir;
(c) implanting the delivery device with a patient so that the septum is beneath and closely adjacent a region of skin of a patient to provide a subcutaneous access site, and locating the distal end of the conduit proximate the determined nerve tissue for releasing medicine to the nerve tissue;
(d) providing an external cannula that is in flow communication with a source of medicine;
(e) extending a distal end of the cannula through the skin of the patient and the elastomeric septum;
(f) releasing medicine from the source of medicine through the cannula into the reservoir and applying sufficient flow pressure so that medicine in an amount effective to provide pain relief is flowed from the reservoir to the distal end of the conduit and released proximate the determined nerve tissue;
(g) monitoring pain experienced by the mammal and determining when the pain is sufficient to indicate the need for additional medicine to be delivered to the determined nerve tissue;
(h) repeating steps (e) and (f) in response to a determination in step (g) that additional pain medicine is needed; and
(i) repeating steps (g) and (h) to treat later experienced pain associated with the determined nerve tissue.

12. The method of claim 10, wherein the step of releasing medicine further comprises removing the source of medicine from the cannula and subsequently providing a saline solution in fluid flow connection with the cannula, and applying sufficient flow pressure to the saline solution wherein the flow pressure of the saline solution pushes at least a portion of the medicine in the reservoir through to the distal end of the conduit to release proximate the determined nerve bundle and provide pain relief.

13. The method of claim 10, wherein the reservoir comprises a titanium reservoir.

14. The method of claim 10, wherein the reservoir comprises a low-profile reservoir.

15. The method of claim 10, wherein the conduit has a plurality of sidewall apertures in the distal portion.

16. The method of claim 10, wherein the conduit comprises a silicone elastomer conduit.

17. The method of claim 10, wherein the medicine comprises a local anesthetic.

18. The method of claim 10, wherein the device may remain implanted within the patient and be used for treating chronic nerve pain for a period of time ranging from about one week to about 50 years.

19. A method for treating chronic nerve pain in a mammalian patient, the method comprising the steps of:
(a) implanting a subcutaneous medicine delivery device beneath a region of skin of the patient, wherein the delivery device comprises a reservoir end comprising a reservoir, a septum, and a distal end comprising a catheter tube having a plurality of sidewall apertures, and wherein the tube is in fluid flow communication with the reservoir, and wherein the distal end is disposed proximal to a desired nerve bundle so that medicine may flow through the sidewall apertures to the nerve bundle, and the reservoir end is disposed distal from the desired nerve bundle to provide a subcutaneous access site;
(b) extending an external cannula in fluid flow communication with a source of medicine through the skin of a patient and the septum into a lumen of the reservoir;
(c) disposing a medicine into the lumen of the reservoir through the cannula by applying fluid flow pressure to the source of medicine, wherein the fluid flow pressure of the medicine pushes at least a portion of the medicine through to the distal end of the tube and out the sidewall apertures to contact at least a portion of the nerve bundle; and
(d) removing the source of medicine from the cannula;
(e) providing a source of saline solution in fluid flow communication with the cannula;
(f) disposing the saline solution into the lumen of the reservoir through the cannula by applying fluid flow pressure to the source of saline, wherein the pressure of the saline pushes at least a portion of the medicine through to the distal end of the tube and out the sidewall apertures to contact at least a portion of the nerve bundle, wherein the medicine provides pain relief to the nerve bundle, and wherein the reservoir is refillable.

20. The method of claim 18 wherein the catheter tube comprises a silicone elastomer catheter tube.

21. The method of claim 18 wherein the catheter tube is tapered from the reservoir end to the distal end, with the reservoir end having a diameter greater than the diameter of the distal end.

22. The method of claim 18 wherein the reservoir comprises a low-profile reservoir.

23. The method of claim 18 wherein the septum comprises an elastomeric septum.
